(19) United States
(12) Patent Application Publication
(10) Pub. No.: US 2026/0174554 A1
(43) Pub. Date: Jun. 25, 2026

(54) PROSTHETIC HEART VALVE DELIVERY SYSTEM AND METHOD

(71) Applicant: Shifamed Holdings, LLC, Campbell, CA (US)

(72) Inventors: Jonathan Hwan OAKDEN, San Jose, CA (US); David Jerry SCOTT, San Mateo, CA (US)

(21) Appl. No.: 19/123,741

(22) PCT Filed: Oct. 26, 2023
(86) PCT No.: PCT/US2023/077962

§ 371 Date: Apr. 23, 2025

Related U.S. Application Data
(60) Provisional application No. 63/381,061, filed on Oct. 26, 2022.

(51) Int. Cl. A61F 2/24

(52) U.S. Cl. CPC A61F 2/2436; A61F 2/2418; A61F 2220/0008

(57) ABSTRACT

Embodiment of a delivery system and method for a prosthetic heart valve are provided herein. The system comprises a valve capsule shaped and size to contain a valve frame in a compressed configuration; a capsule shaft catheter connected to the valve capsule and extending proximally from the valve capsule; and a valve retainer removably connected to the valve frame. The system further comprises one or more elongate, flexible members connected to at least two points at or near a proximal end of the prosthetic heart valve, the elongate flexible member extending from the valve proximal end towards the capsule shaft catheter and configured to apply tension to the valve proximal end. A proximal controller at a proximal end of the capsule shaft catheter can be configured to remove the capsule from the valve frame, thereby permitting the valve frame to expand.

### PRIORITY CLAIM

This application claims the benefit of U.S. Provisional Application No. 63/381,061, filed Oct. 26, 2022, the entire disclosure of which is incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Blood flow between heart chambers is regulated by native valves, i.e., the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully close, causing the patient to experience regurgitation.

While medications may be used to treat diseased native valves, the defective valve may need to be repaired or replaced at some point during the patient's lifetime.

### SUMMARY

In a first aspect, a delivery system for a prosthetic heart valve is provided. The heart valve comprises an anchor adapted to be disposed in a ventricle adjacent a native valve of a patient's heart and a valve frame supporting valve leaflets adapted to be expanded within the anchor. The delivery system comprises a valve capsule, the valve capsule shaped and sized to contain the valve frame within in a compressed configuration; a capsule shaft catheter connected to the valve capsule and extending proximally from the valve capsule; a valve retainer removably connected to the valve frame; one or more elongate, flexible members connected to at least two points at or near a proximal end of the prosthetic heart valve, the elongate flexible member extending from the valve proximal end towards the capsule shaft catheter and configured to apply tension to the valve proximal end; and a proximal controller at a proximal end of the capsule shaft catheter, the proximal controller being configured to remove the capsule from the valve frame, thereby permitting the valve frame to expand.

In some embodiments, the delivery system comprises one or more of the following features. In some embodiments, one or more elongate flexible members comprises suture. The one or more flexible elongate members can be connected to the valve proximal end at two or more points spaced equidistantly around the valve proximal end. In some embodiments, the one or more flexible elongate members are looped around a portion of the valve proximal end at the at least two points. The one or more flexible elongate members can be harnessed to a distal end of another proximally located elongate member that extends proximally through the capsule shaft catheter. In some embodiments, one of the one or more flexible elongate members loops two portions of the valve proximal end. One or more flexible, elongate members can extend proximally through the capsule shaft catheter. In some embodiments, the one or more flexible, elongate members extends proximally to the proximal controller. The proximal controller can be further configured to adjust the applied tension from the elongate flexible members.

In another aspect, a method of delivering a prosthetic heart valve is provided. The method comprises advancing a delivery catheter comprising a valve prosthesis comprising a distal end and a proximal end through a native valve; retracting the valve delivery catheter relative to the valve prosthesis to expose the distal end of the valve prosthesis, thereby expanding the distal end of the valve prosthesis on a first side of the native valve; providing tension to one or more elongate flexible members connected to the proximal end of the valve prosthesis to at least partially constrain radial expansion of the proximal end of the valve, the one or more elongate flexible members extending proximally through the valve delivery catheter; further retracting the valve delivery catheter relative to the valve prosthesis to expose the proximal end of the valve prosthesis on a second side of the native valve; and at least partially releasing the tension from the one or more elongate flexible members, thereby expanding the proximal end of the valve prosthesis.

In some embodiments, the method comprises one or more of the following steps or features. In some embodiments, advancing further comprises advancing the distal end of the delivery catheter through an anchor that is encircling native leaflet(s) and/or chordae within the first side of the native valve. Releasing tension from the one or more elongate flexible members can comprise manipulating a control on a proximal end of the delivery catheter. In some embodiments, manipulating a control on the proximal end of the delivery catheter comprises manipulating a knob or a slide. The method can comprise re-tensioning the one or more elongate flexible members to at least partially collapse the proximal end of the valve prosthesis. In some embodiments, the method comprises retrieving the valve prosthesis. The method can comprise repositioning the valve prosthesis. In some embodiments, the method comprises decoupling the one or more elongate, flexible members from the proximal end of the valve prosthesis. The method can comprise withdrawing the delivery catheter.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A-1B show an implantable valve prosthesis, in accordance with some embodiments.

FIG. 2 and FIG. 3 show embodiments of aspects of a valve delivery subsystem.

FIG. 4 shows an embodiment of the distal end of the valve delivery subsystem.

FIGS. 5A-B show embodiments of details of a tab retainer.

FIG. 6 shows an embodiment of a proximal controller for a valve delivery subsystem.

FIGS. 7A-7G show an embodiment of a method for delivery of a proximal end of a valve using a suture tension mechanism.

FIG. 8 shows an embodiment of a suture harness for connecting to suture connected to the valve proximal end.

FIGS. 9A-9E show an embodiment of a method for delivery of a valve prosthesis using a suture tension mechanism.

FIGS. 10A-10D illustrate another embodiment of a valve delivery subsystem.

### DETAILED DESCRIPTION

This disclosure is directed to a delivery system for a prosthetic heart valve that has two main components: an anchor adapted to be disposed in a ventricle adjacent a native valve of a patient's heart and a valve (e.g., comprising a frame supporting prosthetic valve leaflets) adapted to be delivered after delivery of the anchor and then expanded within the anchor. In particular, the valve is a prosthetic mitral valve, and the delivery system of this invention delivers the valve's two components transeptally. In use, the delivery system advances distally from an entry point in the patient's femoral vein, enters the right atrium of the heart, and passes through the septum into the left atrium to implant the anchor and then expand the valve frame inside the anchor.

The valve can comprise a self-expandable structure configured to expand at or near the implantation site (e.g., the heart valve). Reliably and consistently controlling the expansion of the valve while transitioning from a collapsed state within the delivery system to an expanded state for implantation can help to prevent tissue damage and trauma to tissue around the implantation site. Additionally, while delivering and implanting the valve frame, it may also be necessary to retrieve the valve during implantation if its position is not quite right. The prosthetic valve delivery system of this invention therefore provides systems and methods for retrieving the valve to correct or modify its position and for controllably releasing the valve when it has been correctly placed.

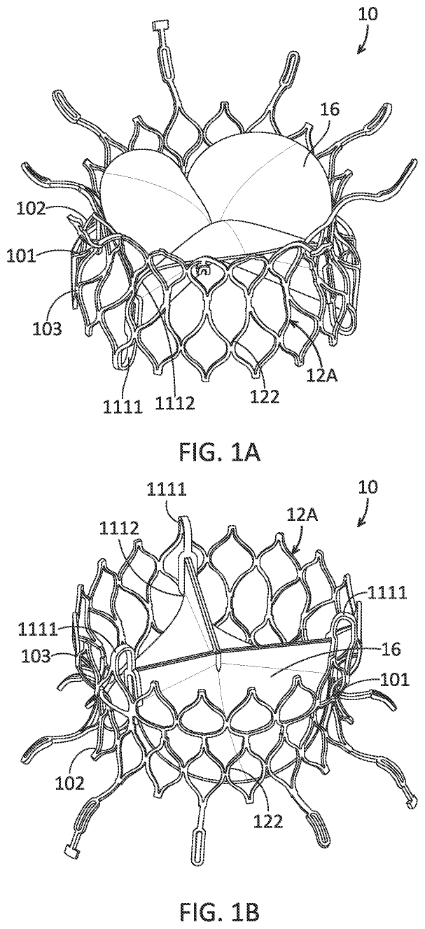

FIGS. 1A-1B show an exemplary valve prosthesis 10 having a valve frame 12a and a plurality of leaflets 16 therein. The valve frame 12a can include interior commissure attachment mechanisms 1111 for attachment of the commissures 1112 of leaflets 16 to the frame structure 12. The valve frame 12a can be deployed from a collapsed (delivery) configuration to an expanded configuration during a method of replacing or repairing a native valve, such as a mitral valve. As shown in FIGS. 1A-1B, the valve frame 12a can include a plurality of rows (e.g., 3-7 rows) of substantially diamond-shaped cells 122. The valve frame 12a can foreshorten during delivery (i.e., as the valve frame 12 transitions from the collapsed configuration to the expanded configuration) due to the cell structure. In some embodiments, the valve frame 12a can be configured to self-expand from the collapsed configuration to the expanded configuration (e.g., can be made of nitinol). The valve frame 12a can provide circumferential strength and/or longitudinal strength to valve prosthesis 10.

The valve prosthesis 10 can be deployed in an expanded configuration according to the methods described herein. For example, valve prosthesis 10 can be deployed into an expanded configuration in a method of replacing or repairing a native anatomical structure. In the expanded configuration, valve prosthesis 10 can be positioned and/or anchored at a target region of a subject (e.g., an organ or tissue of an animal such as a dog, cat, horse, or human). For example, valve prosthesis 10 can be positioned in the expanded configuration in the orifice of a heart valve, such as the mitral valve or tricuspid valve (e.g., to function as a temporary or permanent replacement for an existing mitral valve or tricuspid valve of the heart).

One or more portions of the valve frame 12a can be shaped or configured to aid in securing the valve frame 12 at a location (e.g., in the orifice of a native heart valve). For example, the valve frame 12a can include an atrial flared portion 102 and a ventricular flared portion 103 configured to help secure the frame in the anatomy. The atrial and ventricular flared portions 102, 103 can extend radially outwards from a central circumferential portion 101. The atrial flared portion 102 can, for example, extend into the atrium of the heart from the central circumferential portion 101 when the valve prosthesis is deployed in the native mitral valve. The ventricular flared portion 103, in turn, can extend into the ventricle of the heart from the central circumferential portion 101 when the valve prosthesis is deployed in the native mitral valve. The atrial and ventricular flared portions 102, 103 can, for example, be configured to be positioned on either side of an external (e.g., flat) spiral anchor (e.g., that is wrapped around the chordae) to anchor the valve frame 12 in the anatomy. Alternatively or additionally, the atrial and ventricular flared portions 102, 103 can be configured to engage with tissue to prevent the valve prosthesis from slipping through the native valve orifice.

Exemplary methods for delivery of the anchor and valve are provided in U.S. Provisional Patent Application No. 63/366,115, filed Jun. 9, 2022, the entire disclosure of which is incorporated by reference herein.

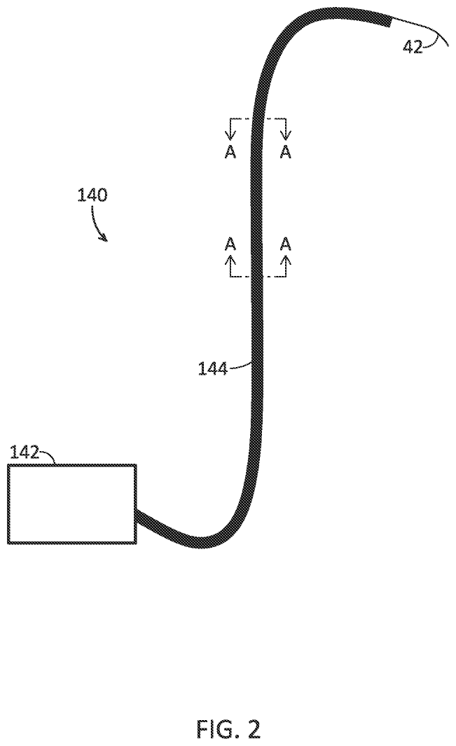

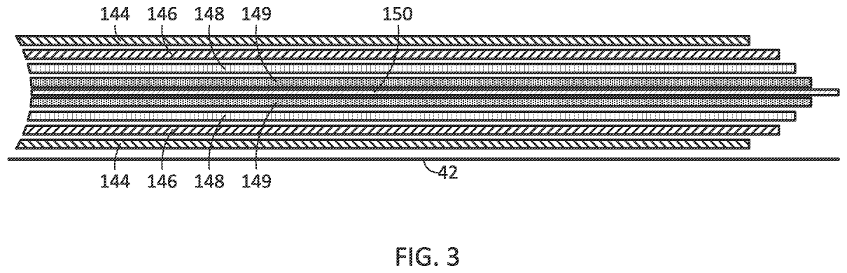

FIG. 2 and FIG. 3 show aspects of a valve delivery subsystem 140 having a proximal controller 142 and five nested catheters (as shown in cross-section in FIG. 3): An outer steerable catheter 144, a capsule shaft 146 movably disposed in the lumen of the outer steerable catheter 144, and an inner steerable catheter 148 movably disposed in the lumen of the capsule shaft. As shown, the tether 42, which is still extending proximally from the previously-implanted anchor, is disposed outside of outer steerable catheter 144. In some embodiments, the tether 42 is disposed within the outer steerable catheter 144 but outside of the capsule shaft 146. In some embodiments, the tether 42 is removed prior to introduction and advancement of the valve delivery subsystem 140. Disposed within the lumen of inner steerable catheter 148 is a tab retainer shaft 149, and disposed within the lumen of tab retain shaft 149 is a nose cone shaft 150. A guidewire (not shown) may be disposed in a lumen of nose cone shaft 150. Outer steerable catheter 144 and inner steerable catheter 148 may be configured like the outer steerable catheter and inner steerable catheter described above with respect to the anchor delivery subsystem.

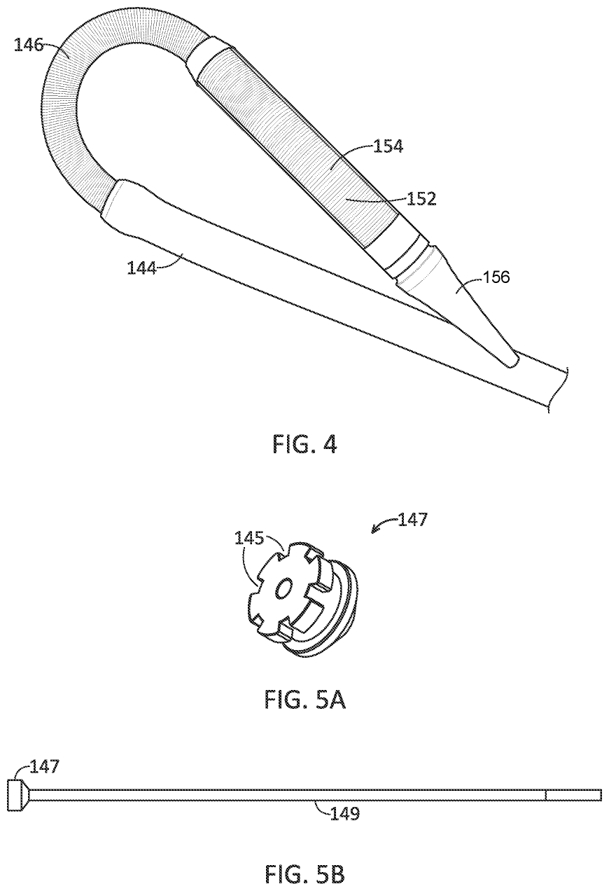

FIG. 4 shows the distal end of the valve delivery subsystem, and FIGS. 5A-5B show details of the tab retainer and shaft. In the view of FIG. 4, capsule shaft 146 is shown extending distally of the distal end of outer steerable catheter 144. A valve capsule 152 containing a compressed prosthetic valve 154 is attached to the distal end of the capsule shaft 146. When loaded in the capsule 152, tabs (not shown) on the proximal (atrial) end of valve 154 are disposed in slots 145 of a tab retainer 147 at the distal end of tab retainer shaft 149. A nose cone 156 extends from the distal end of capsule 152. Nose cone 156 is connected to the nose cone shaft 150 (not shown in FIG. 4).

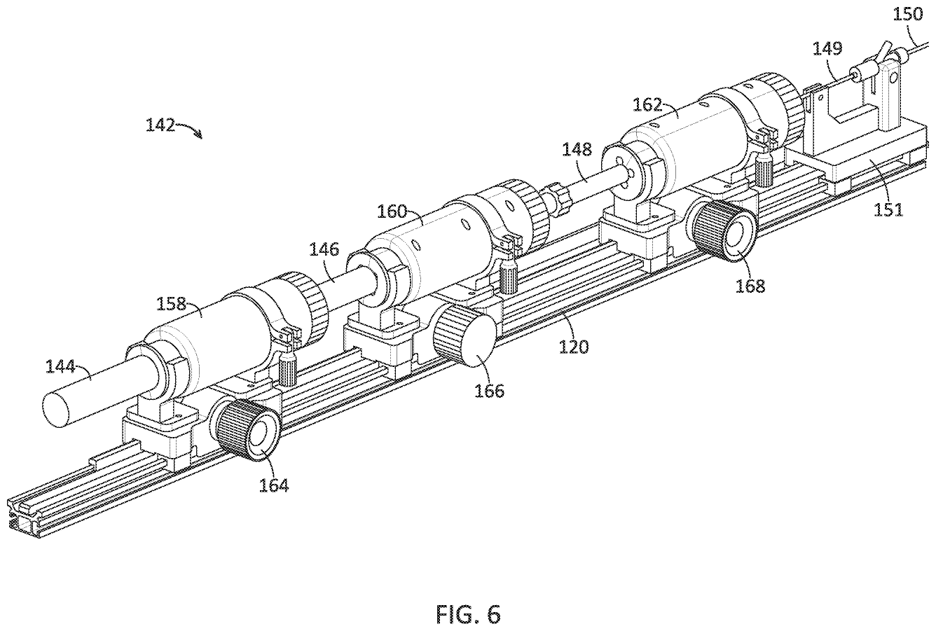

FIG. 6 shows the proximal controller 142 for the valve delivery subsystem. The tab retainer shaft 149 and nose cone shaft 150 within it are disposed on a carriage 151 mounted on a rail 120 at the proximal end of proximal controller 142. The guidewire described above may be inserted into nose cone shaft 150. Control handles 158, 160, and 162 for the outer steerable catheter 144, capsule shaft 146, and inner steerable catheter 148 are also movably mounted on rail 120. Rotational knobs 164, 166, and 168 advance and retract the control handles along the rail 120, and can be locked in place when they are not being moved.

The valve delivery subsystem is placed into the patient's vasculature through the same femoral vein introducer sheath used for the anchor delivery and implantation. To advance the valve capsule to the heart, the control handles 158, 160, and 162, and carriage 151, are advanced together along rail 120 under fluoroscopic guidance. During navigation of the prosthetic valve through the vasculature, the distal end of the valve delivery subsystem is steered by bending the distal ends of inner and outer steering catheters 148 and 144 using control handles 162 and 158, respectively, as described above with respect to the inner and outer steering catheters of the anchor delivery subsystem. The valve capsule 152 and nose cone 156 are just distal to the distal end of the outer steerable catheter 144 during advancement into the patient's heart.

When the nose cone 156, valve capsule 152, and the distal end of the outer steerable catheter 144 have passed through the septum into the left atrium of the heart, the valve capsule shaft 146, the inner steerable catheter 148 and valve capsule 152 are advanced out of the outer steerable catheter by moving control handles 160 and 162 and carriage 151 distally while keeping control handle 158 stationary. The above are then steered and further advanced through the native valve annulus to move the valve capsule 152 into position within the previously implanted anchor. Once in position, the capsule shaft 146 may be retracted while keeping the tab retainer shaft 149 and valve 154 stationary to retract capsule 152 and expose the distal end of valve 154, thereby allowing it to begin to self-expand within the anchor. The partially self-expanded valve may be pulled proximally against the anchor to move the valve and anchor closer to (e.g., the ventricular side of) the native valve annulus. Thereafter, the capsule shaft is retracted further to expose the proximal end of valve 154 to allow it to fully self-expand within the atrium. When the capsule 152 has been retracted sufficiently to expose the slots 145 of tab retainer 147, the tabs on the valve move out of the slots 145 to release the valve 154 from the tab retainer 147. The valve delivery subsystem may then be removed from the patient.

In some embodiments, the method of delivering the valve comprises securing one or more flexible, elongate members (e.g., suture, wire, etc.) to portions of a proximal portion of the valve. The flexible, elongate members can be connected to one or more portions at or near a proximal end of the valve. The flexible, elongate members can be tensioned, keeping proximally directed tension on the connected portion of the valve. Greater control over the atrial side of the valve may be desired as the atrial side of the valve has a greater tendency to spring open and out of the delivery system than the ventricular side. This greater likelihood of the atrial side to spring open is due, at least in part, to retraction of the delivery catheter relative to the atrial side moving the atrial side closer to maximal expansion, instead of away from maximal expansion as with retraction of the delivery catheter relative to the ventricular side.

This tension and control over the proximal end of the valve can allow the capsule to be retracted past the proximal portion of the valve without causing sudden expansion of the proximal portion of the valve. Additionally, the tension can help prevent the valve from fully deploying in an incorrect location, such as the ventricle, which may have catastrophic results.

While a particular embodiment of a flexible, elongate member (e.g., suture) may be used in the description, it will be appreciated that other flexible, elongate members are also possible.

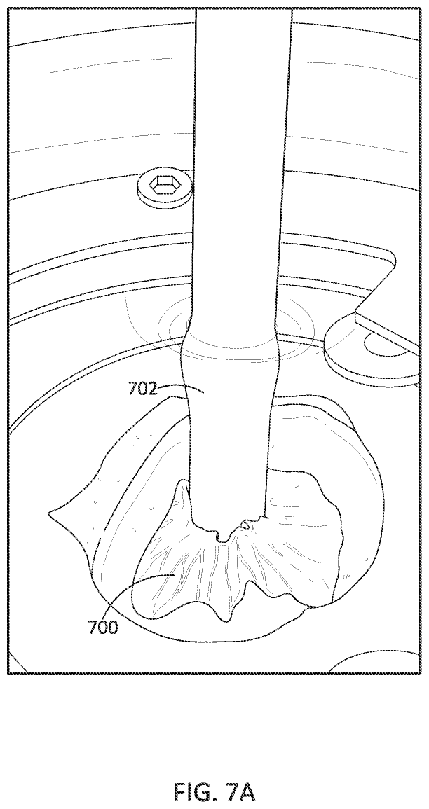

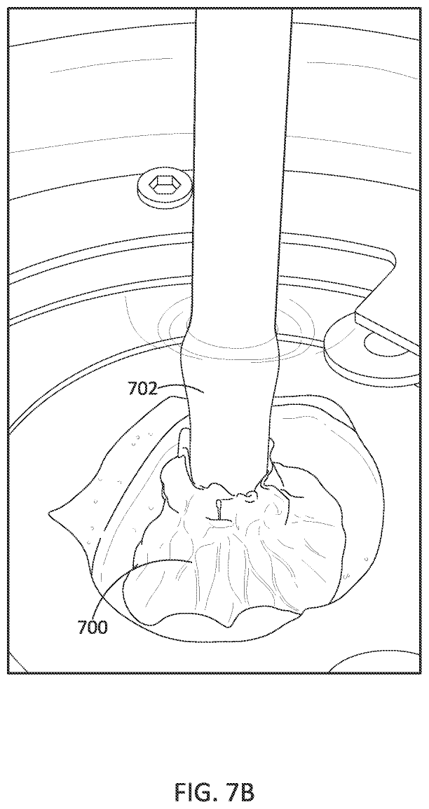

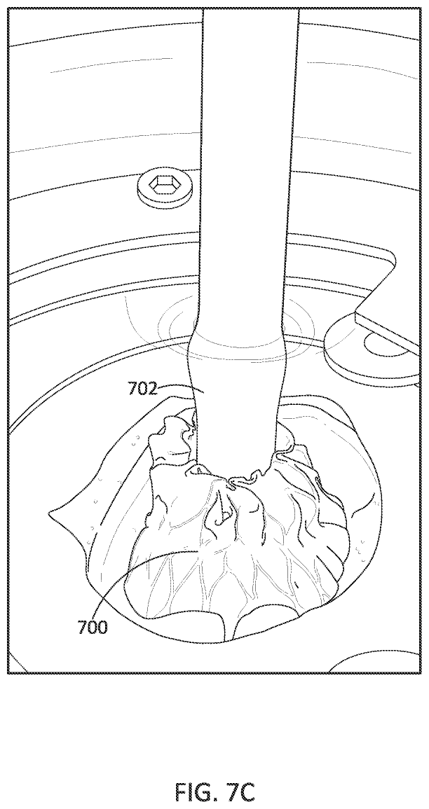

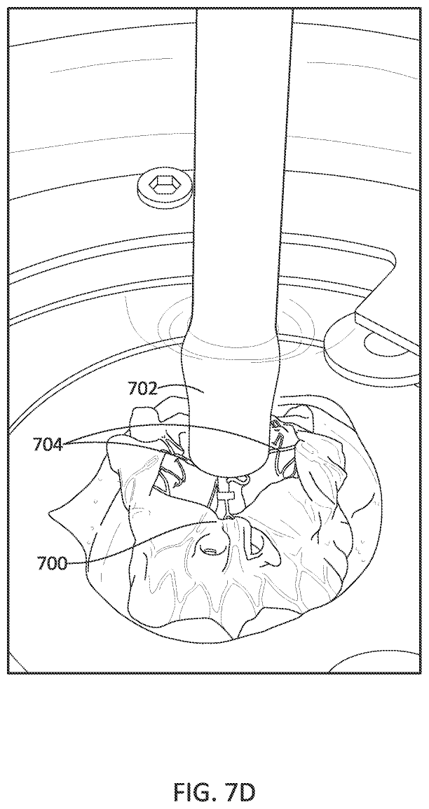

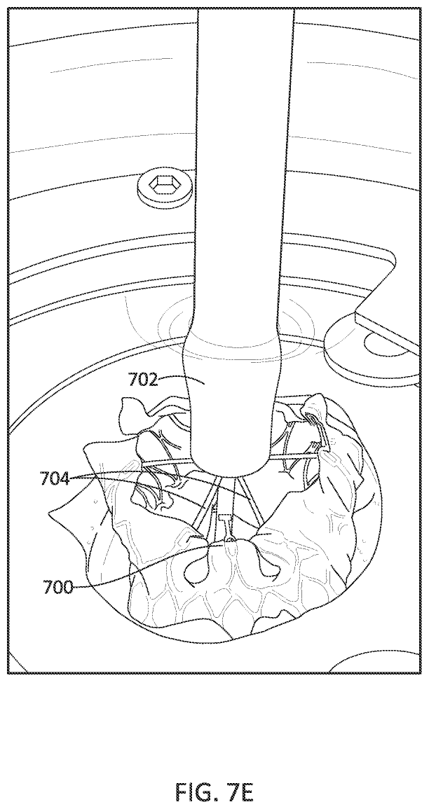

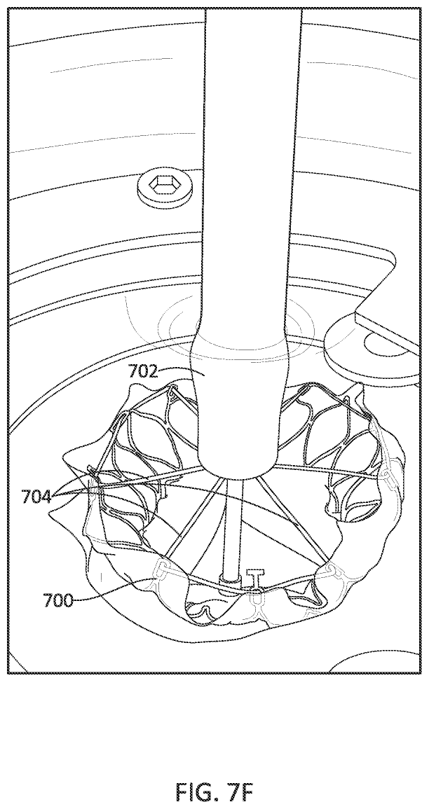

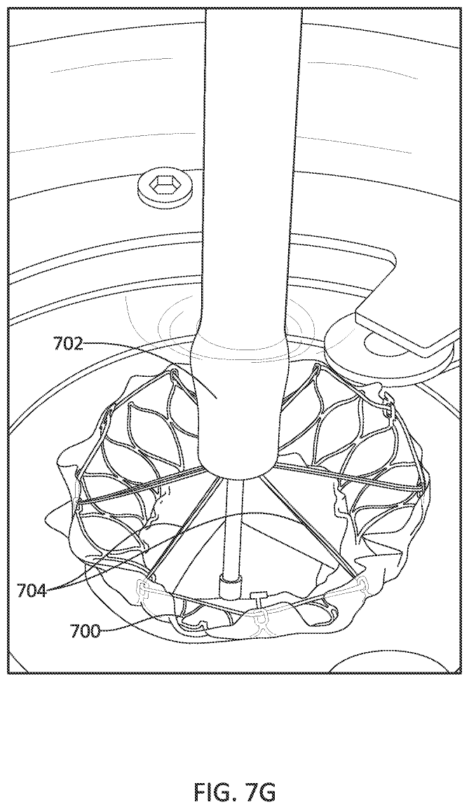

FIGS. 7A-7G depict an exemplary method of delivering a valve prosthesis 700 (similar to, e.g., valve prosthesis 10, valve prosthesis 154). The valve prosthesis may be delivered by retracting the capsule shaft to expose the valve, as described above. FIGS. 7A-7G do not show the valve capsule, and instead show just a generic delivery shaft.

As shown in FIG. 7A, the delivery shaft 702 may be exposed sufficiently for the distal end of the valve 700 to be exposed from the shaft 702 and expanded.

As shown in FIG. 7B, the shaft 702 may be retracted relative to the valve 700 to begin to expose the proximal end of the valve 700.

Moving to FIG. 7C, the shaft 702 is further retracted relative to the valve 700, causing more of the proximal end of the valve 700 to be exposed and to expand.

FIG. 7D shows the shaft 702 sufficiently retracted to expose the entire valve 700. The valve 700 is partially expanded relative to its position within the shaft; however, the valve 700 is held in a partially collapsed configuration by flexible, elongate members (e.g., suture) 704 extending from a distal end of the shaft 702 and connecting to a variety of locations around a proximal end of the valve 700.

FIGS. 7E and 7F show the shaft 702 retracted as shown in FIG. 7D, but also showing the suture 704 under reduced tension as compared to the previous figure. This reduced tension allows the valve 700 to continue its expansion.

FIG. 7G shows the suture 704 given enough slack to allow full expansion of the valve 700.

At this point, the suture 704 may be re-tensioned to allow recapture of the valve prosthesis for removal or repositioning. Once the clinician is satisfied with the positioning of the valve prosthesis, the suture may be removed or disconnected from the valve prosthesis and may be withdrawn, leaving the implanted valve prosthesis in place.

In some embodiments, the suture is attached to the valve prosthesis by looping it through a portion of the valve. For example, the suture may be looped through a crown of the proximal end of the valve. Other connection locations are also contemplated.

In some embodiments, the portion of the valve prosthesis through which the suture is looped comprises a component, such as a cleat, holding the loop in place and/or preventing it from being pulled through the valve portion. After desired positioning and expansion of the valve prosthesis, the cleat may be removed, allowing withdrawal of the sutures. Removal of the cleat may be actuatable using a control in the handle.

In some embodiments, a suture may be connected or looped to two or more locations or connection points (e.g., 2, 3, 4, 5, 6, 7 or more, etc.) around the proximal end of the valve. The two or more locations may be equally spaced about the proximal end of the valve to allow for a balanced tensioning of the proximal end of the valve from the suture. For example, there may be three connection points spaced equidistantly around the valve proximal end.

In some embodiments, the suture may be connected to each crown surrounding the valve proximal end. In some embodiments, the suture may be connected/looped to every other crown surrounding the valve proximal end. Other configurations are also contemplated (e.g., every 3 crowns, every 4 crowns, etc.).

In some embodiments, each suture and/or suture loop connected to the valve proximal end extends proximally through the valve delivery catheter to the handle.

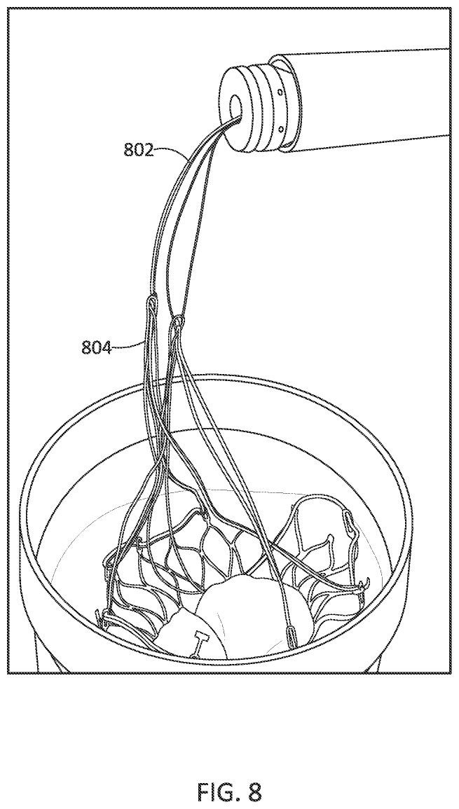

In some embodiments, the delivery system may comprise one or more master sutures and/or suture loops extending from or through the valve delivery catheter. As shown in FIG. 8, each of the master sutures 802 may be connected or harnessed to distally located sutures/suture loops 804 connecting to more than one location around a proximal end of the valve. For example, suture/suture loops may extend proximally from multiple connection points at the valve proximal end and may all connect to a single master suture extending through the valve delivery catheter to the handle. For another example, there may be six total connection points around the valve proximal end, each of which comprises suture extending proximally and connecting to or joining suture from an adjacent connection point, resulting in three secondary connection points with three master suture/suture loops extending from or through the valve delivery catheter.

In some embodiments, a single suture may loop around a portion of the proximal end (e.g., a crown) and then extend proximally towards the valve delivery catheter. In some embodiments, a suture may loop through multiple portions of the valve proximal end before extending proximally towards the valve delivery catheter. For example, the suture may loop through two portions of the valve proximal end before extending away from the valve proximal end and towards the valve delivery catheter. For another example, the suture may loop through three portions of the valve proximal end before extending towards the valve delivery catheter.

In some embodiments, the suture extending through the valve delivery catheter are tensioned using a control on the handle. The control can allow the tension holding the valve in a collapsed position to be fixed, for example, by clamping the elongate, flexible members extending through the delivery system proximal end. A stopcock type mechanism, with an opening and a clamp can be used to clamp the suture as well as adjust tension by allowing the suture to be pulled proximally or released distally prior to clamping.

In some embodiments, releasing the tension on the suture can allow the valve to expand at a desired rate from a balance of the expansion force of the valve and the friction from the suture loops and within the delivery system.

In some embodiments, releasing the tension on the suture can be controlled using, for example, a knob or slide on the handle of the delivery system.

The amount of friction force applied by the suture loops can depend on a number of features including the looping pattern and the height of the connection points relative to the distal end of the capsule shaft. Increasing the surface area of the suture connected or looped through the valve can increase the friction force exerted against the expansion force of the valve. For example, a delivery system comprising a greater number of suture loops will experience more frictional force than a delivery system comprising a lower number of suture loops.

Additionally, increasing the height between the distal end of the capsule sheath and the proximal end of the valve can reduce the frictional force exerted by the sutures against the expansion force of the valve. In some embodiments, though, the distal end of the capsule sheath will be kept positioned just proximal of the proximal end of the valve.

In some embodiments, a delivery system including suture connected to the proximal end of the valve may utilize an embodiment of a tab retainer that is different from that shown in FIGS. 5A and 5B. The tab retainer in FIGS. 5A and 5B can receive the tabs of the valve in the slots 145 and the shelf. These features allow the tab retainer to be used to apply tension to or proximally retract the valve proximal end when the capsule shaft is positioned over the tabs. In embodiments of a delivery system including the suture connection to the valve proximal end, these features of the tab retainer are not needed as the suture can be used to apply tension to or proximally retract the proximal end of the valve. In such embodiments, the tab retainer may only comprise a shelf, without the slots 145 shown in FIG. 5A.

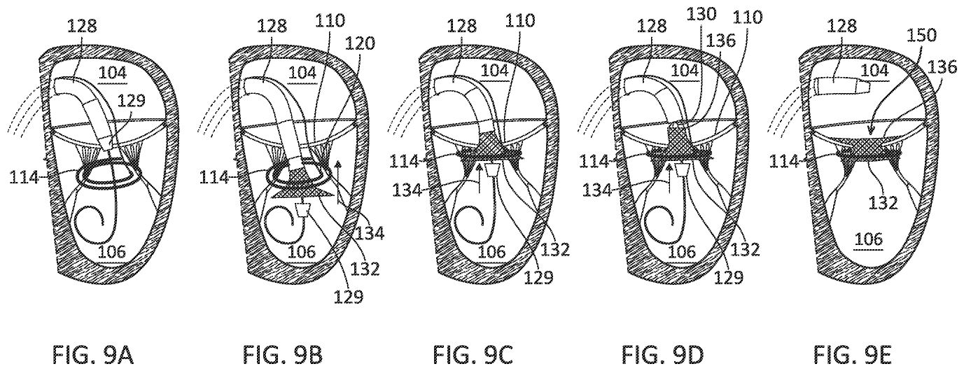

An embodiment of a method of delivery the valve proximal end using the elongate, flexible members is shown in FIGS. 9A-9E. The method comprises advancing the valve capsule sheath 128 and nosecone 129 through the atrium 104 and the annulus 110 toward the ventricle 106, as shown in FIG. 9A.

Following further advancement of the valve capsule sheath 128 and nosecone 129 through the annulus 110 and anchor 114, and into the ventricle 106, the valve capsule sheath 128 is retracted to expose the distal end 132 of the valve, as shown in FIG. 9B. The nosecone 129 is shown distal to the valve prosthesis.

At this point, traction can be applied to the valve delivery catheter to seat the anchor 114 close to the annulus. The ventricular flare of the valve prosthesis may enable this traction, as shown in FIG. 9C. In some embodiments, the entire valve delivery catheter is retracted to adjust the position of the anchor 114. In some embodiments, selected inner portions of the valve delivery catheter are retracted to adjust the position of the anchor 114, while other portions remain in place. For example, inner portions such as the valve capsule sheath 128, inner steerable catheter, and tab retainer catheter can be retracted while the outer sheath and nosecone remain in place.

During delivery of the valve, the elongate, flexible member(s) (e.g., sutures) extends proximally from and is connected to the valve proximal end and is applying tension to the valve proximal end. As shown in FIG. 9D, the valve capsule sheath 128 is further retracted to expose the proximal end 136 of the valve prosthesis, while suture(s) 130 are attached to the valve proximal end 136 and under tension. Proximally-directed (134) tension applied to the suture 130 (e.g., at a valve delivery system control) constrains (e.g., radial) expansion of the valve proximal end 136. As shown in FIG. 9D, the proximally directed tension is preventing the valve proximal end 136 from fully expanding.

Tension on the suture 130 may then be (e.g., controllably) released to allow the atrial brim of proximal end 136 to expand. In some embodiments, the tension release of the suture(s) 130 enables preservation of the axial height of the valve delivery catheter during valve deployment. Once the valve is in a desired position, the elongate, flexible member 130 may then be decoupled from the valve prosthesis, fully releasing the valve prosthesis. The delivery system may then be withdrawn, leaving the valve prosthesis 150 in place within the anchor 114 and across the native valve, as shown in FIG. 9E.

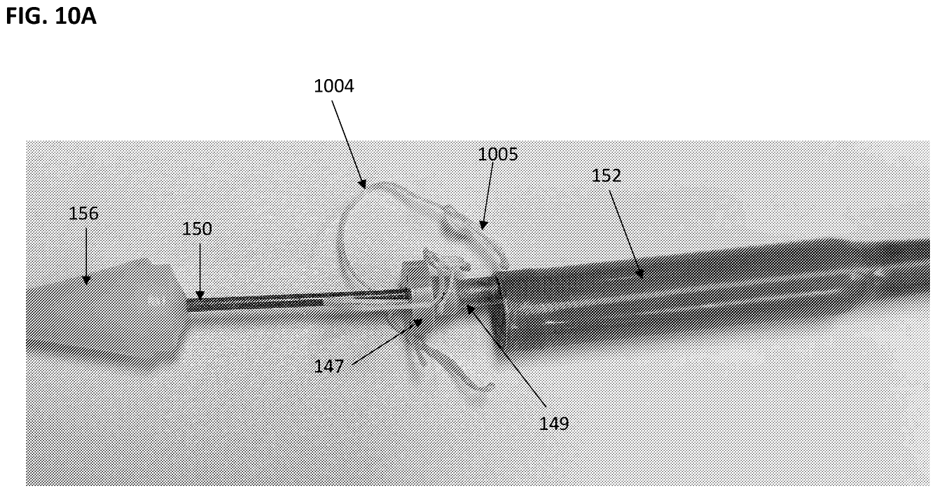

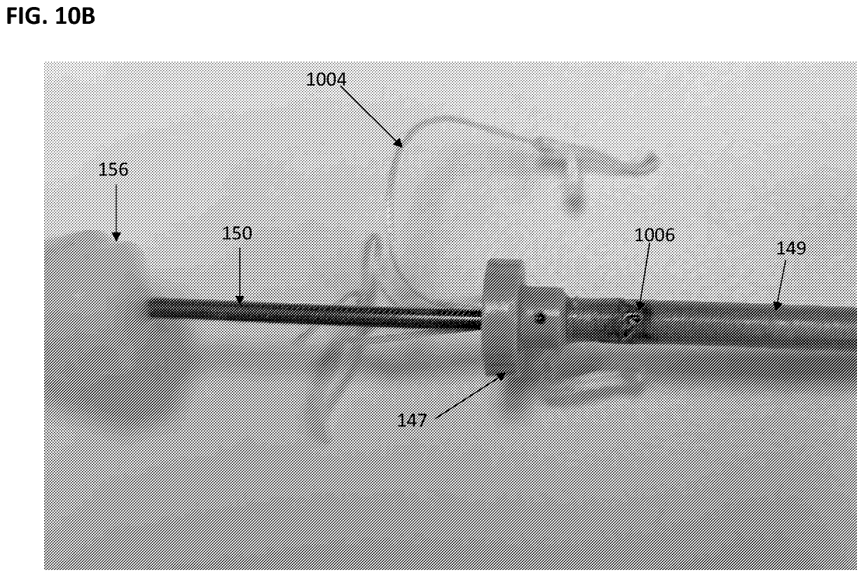

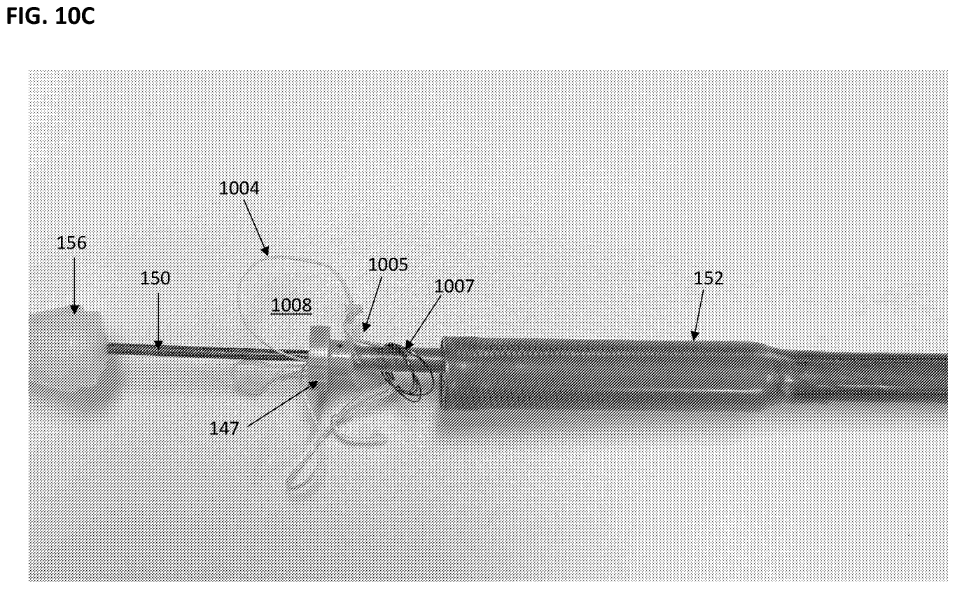

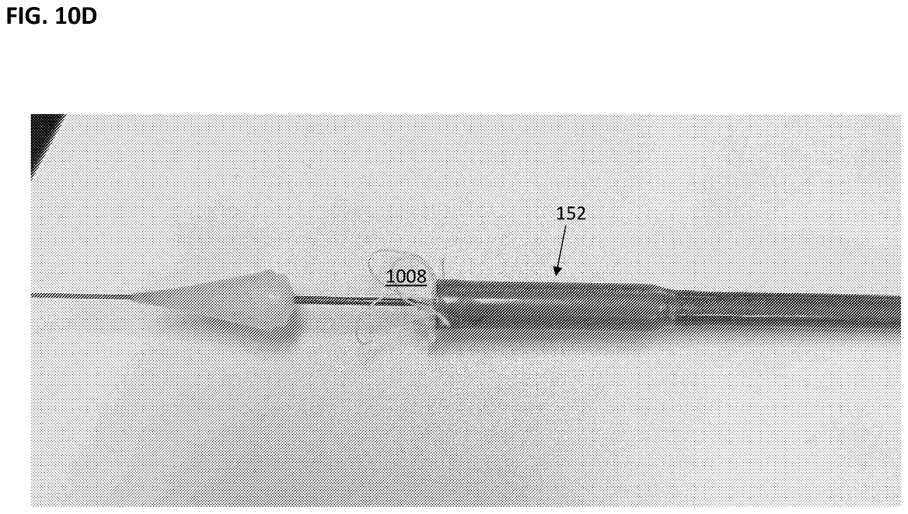

FIG. 10A is another embodiment of the distal end of the valve delivery subsystem, including valve capsule 152, tab retainer 147, tab retainer shaft 149, nose cone shaft 150, and nose cone 156. One or more sutures 1004 are shown extending out of the valve delivery subsystem from the annular space between nose cone shaft 150 and tab retainer shaft 149, distal to the tab retainer 147. The suture(s) 1004 can include a distal end 1005 that terminates in a loop, as shown. The sutures can extend proximally back in the valve delivery subsystem to a handle or control where they can be manipulated (e.g., advanced or retracted) by a user.

FIG. 10B is another view of the distal end of the valve delivery subsystem, with the valve capsule pulled back proximally to reveal an exit port 1006 passing through the tab retainer shaft to expose the annular space between the tab retainer shaft and the nose cone shaft.

In FIG. 10C, a shape memory (e.g., nitinol) wire 1007 is shown exiting the exit port in the tab retainer shaft. The shape memory wire can extend proximally back in the valve delivery subsystem to a handle or control where they can be manipulated (e.g., advanced or retracted) by a user. The shape memory wire 1007 is configured to assume a shape memory configuration (e.g., a loop) when it exits the exit port. As shown, the shape memory wire has assumed a plurality of loops around the tab retainer shaft 149, however it should be understood that only a single loop (or even less) is sufficient. The shape memory configuration of the shape memory wire is configured to loop through or engage with the distal loops 1005 of the sutures 1004, causing the sutures to form a second loop 1008 between where the sutures exit the tab retainer 147 and where the sutures are attached or coupled to the shape memory wire 1007.

FIG. 10D shows the valve capsule 152 advanced over the tab retainer to cause the distal ends of the sutures to be locked or stored into place within the valve capsule, forming loops 1008 in the sutures. In this configuration, the sutures cannot be pulled out of the valve capsule, as they are held by the shape memory wire and also prevented from being pulled out by the tab retainer. Tension on the sutures can be controlled by a user. For example, the sutures can be released to release tension on the sutures and increase a size of loops 1008, or the sutures can be pulled to increase tension on the sutures and decrease a size of loops 1008.

Valve delivery of a valve with the valve delivery subsystem can be performed with the system of FIGS. 10A-10D. In one example, the valve frame can be loaded onto the delivery subsystem, sutures can be passed through the valve, and the shape memory wire can be passed through or affixed to distal ends or loops of the sutures. The valve can the be advanced to the delivery site as previously described herein. Tension on the sutures can be controlled by the user as described in FIG. 10D, by increasing or reducing tension on the sutures exiting near the tab release to increase or decrease a size of the loops formed in the sutures. Tension control of the sutures allows for controlled expansion of the valve frame.

When the valve frame is appropriately positioned and expanded, the sutures can be released from the frame by retracting valve capsule 152 from the tab retainer 147 and pulling shape memory wire 1007 into the tab retainer shaft 149 to release the suture distal end loops from the shape memory wire. The sutures can then be completely released from the valve frame either by pulling the sutures into the delivery device, or removing the entire delivery device from the patient.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A delivery system for a prosthetic heart valve, the prosthetic heart valve comprising an anchor adapted to be disposed in a ventricle adjacent a native valve of a patient's heart and a valve frame supporting valve leaflets adapted to be expanded within the anchor, the delivery system comprising:
a valve capsule, the valve capsule shaped and sized to contain the valve frame within in a compressed configuration;
a capsule shaft catheter connected to the valve capsule and extending proximally from the valve capsule;
a valve retainer removably connected to the valve frame;
one or more elongate, flexible members connected to at least two points at or near a proximal end of the prosthetic heart valve, the elongate flexible member extending from the valve proximal end towards the capsule shaft catheter and configured to apply tension to the valve proximal end; and
a proximal controller at a proximal end of the capsule shaft catheter, the proximal controller being configured to remove the capsule from the valve frame, thereby permitting the valve frame to expand.

2. The delivery system of claim 1, wherein the one or more elongate flexible members comprises suture.

3. The delivery system of claim 1, wherein the one or more flexible elongate members are connected to the valve proximal end at two or more points spaced equidistantly around the valve proximal end.

4. The delivery system of claim 1, wherein the one or more flexible elongate members are looped around a portion of the valve proximal end at the at least two points.

5. The delivery system of claim 1, wherein the one or more flexible elongate members is harnessed to a distal end of another proximally located elongate member that extends proximally through the capsule shaft catheter.

6. The delivery system of claim 1, wherein the one or more flexible elongate members comprises a first plurality of flexible elongate members connected to a valve proximal end and harnessed to a distal end of a first proximally located master elongate member and a second plurality of flexible elongate members connected to a valve proximal end and harnessed to a distal end of a second proximally located master elongate member, the first and second proximally located master elongate members extending proximally through the capsule shaft catheter.

7. The delivery system of claim 6, further comprising a third plurality of flexible elongate members connected to a valve proximal end and harnessed to a distal end of a third proximally located master elongate member, the third proximally located master elongate member extending proximally through the capsule shaft catheter.

8. The delivery system of claim 1, wherein one of the one or more flexible elongate members loops two portions of the valve proximal end.

9. The delivery catheter of claim 1, wherein the one or more flexible, elongate members extends proximally through the capsule shaft catheter.

10. The delivery catheter of claim 9, wherein the one or more flexible, elongate members extends proximally to the proximal controller.

11. The delivery catheter of claim 10, wherein the proximal controller is further configured to adjust the applied tension from the elongate flexible members.

12. The delivery catheter of claim 1, wherein the at least two points comprise at least two crown portions.

13. The delivery catheter of claim 1, wherein the one or more flexible, elongate members are connected to 3, 4, 5, 6, or 7 points at or near a proximal end of the prosthetic heart valve.

14. The delivery catheter of claim 1, wherein the one or more elongate flexible members are connected to the valve proximal end through a removable component configured to hold the elongate flexible member in place relative to the valve proximal end and configured to be removed after proper positioning of the prosthetic heart valve.

15. A method of delivering a prosthetic heart valve, the method comprising:
advancing a delivery catheter comprising a valve prosthesis comprising a distal end and a proximal end through a native valve;
retracting the valve delivery catheter relative to the valve prosthesis to expose the distal end of the valve prosthesis, thereby expanding the distal end of the valve prosthesis on a first side of the native valve;
providing tension to one or more elongate flexible members connected to the proximal end of the valve prosthesis to at least partially constrain radial expansion of the proximal end of the valve, the one or more elongate flexible members extending proximally through the valve delivery catheter;
further retracting the valve delivery catheter relative to the valve prosthesis to expose the proximal end of the valve prosthesis on a second side of the native valve; and
at least partially releasing the tension from the one or more elongate flexible members, thereby expanding the proximal end of the valve prosthesis.

16. The method of claim 15, wherein advancing further comprises advancing the distal end of the delivery catheter through an anchor that is encircling native leaflet(s) and/or chordae within the first side of the native valve.

17. The method of claim 15, wherein releasing tension from the one or more elongate flexible members comprises manipulating a control on a proximal end of the delivery catheter.

18. The method of claim 17, wherein manipulating a control on the proximal end of the delivery catheter comprises manipulating a knob or a slide.

19. The method of claim 15, further comprising re-tensioning the one or more elongate flexible members to at least partially collapse the proximal end of the valve prosthesis.

20. The method of claim 19, further comprising retrieving the valve prosthesis.

21. The method of claim 19, further comprising repositioning the valve prosthesis.

22. The method of claim 15, further comprising decoupling the one or more elongate, flexible members from the proximal end of the valve prosthesis.

23. The method of claim 15, further comprising withdrawing the delivery catheter.

24. The method of claim 15, wherein providing tension to one or more elongate flexible members connected to the proximal end of the valve prosthesis comprises providing tension to a first plurality of flexible elongate members connected to the proximal end of the valve prosthesis and harnessed to a distal end of a first proximally located master elongate member and a second plurality of flexible elongate members connected to a proximal end of the valve prosthesis and harnessed to a distal end of a second proximally located master elongate member, the first and second proximally located master elongate members extending proximally through the capsule shaft catheter.

25. The method of claim 24 wherein providing tension to one or more elongate flexible members connected to the proximal end of the valve prosthesis comprises providing tension to a third plurality of flexible elongate members connected to the proximal end of the valve prosthesis and harnessed to a distal end of a third proximally located master elongate member, the third proximally located master elongate member extending proximally through the capsule shaft catheter.

26. (canceled)
